(19) United States
(12) Patent Application Publication
(10) Pub. No.: US 2023/0133668 A1
(43) Pub. Date: May 4, 2023

(54) SYSTEM AND METHOD FOR TREATMENT WITH NITRIC OXIDE

(71) Applicant: ORIGIN, INC., Princeton, NJ (US)

(72) Inventors: Kirill Gutsol, Warminster, PA (US); Alex Dolgopolsky, Richboro, PA (US); Michael Preston, Orangeburg, NY (US)

(73) Assignee: ORIGIN, INC., Princeton, NJ (US)

(21) Appl. No.: 17/913,409

(22) PCT Filed: Mar. 18, 2021
(86) PCT No.: PCT/US2021/022913

§ 371 Date: Sep. 21, 2022

Related U.S. Application Data
(60) Provisional application No. 62/993,439, filed on Mar. 23, 2020.

(51) Int. Cl. A61M 16/08; A61M 16/12

(52) U.S. Cl. CPC A61M 16/0808; A61M 16/125

(57) ABSTRACT

A system for providing a NO-containing gas flow to treat a biological object. The system includes a nozzle receptacle for receiving NO-rich air from a plasma-generated NO source, tubing coupled to the nozzle for directing the NO-rich air to a scrubber, the scrubber configured to receive a solvent for absorbing NO2, tubing coupled between the scrubber and a gas mixer for directing scrubbed NO-rich air to the gas mixer, where the gas mixer is coupled to a source of atmospheric air for selectively mixing the scrubbed NO-rich air with the atmospheric air to create diluted NO-containing air; and a manifold for distributing the diluted NO-containing air to a plurality of patient locations.

### CROSS-REFERENCE TO RELATED APPLICATIONS

This is an international application claiming priority to pending U.S. Provisional Patent Application Ser. No. 62/993,439, filed Mar. 23, 2020, the entire contents of which provisional patent application is incorporated herein by reference.

### FIELD OF THE DISCLOSURE

The present disclosure generally relates to medical equipment and more specifically to devices and methods for providing treatment of a biological object with mixed gases containing nitric oxide (NO). The disclosed system and methods may be suitable for treating various pathological processes such as viral and bacterial respiratory tract infections, or in any application where inhalation of or intubation with NO is or could be intrinsic to the treatment or a necessary or potential consequence of treatment.

### BACKGROUND OF THE DISCLOSURE

Nitric oxide (NO) is a signaling molecule that has well-established abilities to control blood-flow, control inflammation, control infections from bacteria, viruses and fungi, stimulate tissue- and collagen-regeneration, and mobilize stem-cell migration

Plasma-generated NO—the present inventors have developed a system and method for producing NO by means of a high-energy plasma stream which has shown greater effectiveness in controlling blood-flow, inflammation, and infection than that reported by the use of gaseous NO. An example of the inventors' system and method can be found in U.S. Patent Application Publication No. 2018/0161750A1, titled “Device and Method for Producing High Concentration, Low Temperature Nitric Oxide,” filed Dec. 13, 2017, the entire contents of which is incorporated herein by reference.

NO and respiratory diseases—In a paper originally published in 2005 in connection with the SARS outbreak, and re-released in 2020 by the National Institutes of Health, it was found that that an organic NO donor, S-nitroso-N-acetylpenicillamine, significantly inhibited the replication cycle of SARS CoV in a concentration-dependent manner. It was also shown that NO inhibits viral protein and RNA synthesis. Furthermore, it was demonstrated that NO generated by inducible nitric oxide synthase, an enzyme that produces NO, inhibits the SARS CoV replication cycle.

In view of the foregoing, it would be desirable to provide an improved system and method that allow for the safe and efficacious application of NO to parts of the respiratory system where infections such as coronaviruses reside. While there are physiological impediments to introducing NO into those parts of the lower lung where certain infections establish themselves, the inventors believe that NO's well-established anti-viral and anti-bacterial properties have potential clinical utility in the respiratory system where access is not occluded and, in the case of coronaviruses, before they establish themselves in the lower lung.

Viruses, in particular the coronavirus, target the epithelial cells of the respiratory tract, resulting in diffuse alveolar damage resulting in vascular injury to the lung. This condition creates a structural abnormality in the blood vessel base that is accompanied by hypoxia and an inflammatory response. Nitric oxide modulates vascular injury and interrupts the inflammatory effect of viruses, in particular the coronavirus, selectively. This is clearly the beneficial effect of NO on viral infections in the lungs.

### SUMMARY

A system is disclosed for providing a NO-containing gas flow to treat a biological object. The system can include a nozzle receptacle for receiving NO-rich air from a plasma-generated NO source and tubing coupled to the nozzle for directing the NO-rich air to a scrubber. The scrubber may be configured to receive a solvent for absorbing NO₂. Tubing may be coupled between the scrubber and a gas mixer for directing scrubbed NO-rich air to the gas mixer. The gas mixer may be coupled to a source of atmospheric air for selectively mixing the scrubbed NO-rich air with the atmospheric air to create diluted NO-containing air. A manifold may be provided for distributing the diluted NO-containing air to a plurality of patient locations.

The system may further include a cooling loop for cooling the solvent in the scrubber. The cooling loop may comprise a pump and a heat exchanger. A condenser may be provided for dehumidifying the scrubbed NO-rich air discharged from the scrubber. A concentration monitor may be coupled to the manifold for measuring a concentration of NO in the diluted NO-containing air in the manifold.

In some embodiments the NO-rich air from the plasma-generated NO source may be provided at a flow rate of from 1 to 10 L/min and a temperature of from 300° C. to 1000° C. In some embodiments, the scrubbed NO-rich air may be provided at a flow rate of from 1 to 10 L/min and a temperature of from 25° C. to 40° C. In some embodiments, the dilute NO-containing air may be provided at a flow rate of up to 1200 L/min and at ambient room temperature.

The system may further include a feedback loop coupled between the concentration monitor and the gas mixer. The feedback loop may be for adjusting the gas mixture to adjust an amount of atmospheric air to mix with the scrubbed NO-rich air. The system may further comprise a plurality of valves coupled to the manifold, each of the plurality of valves coupleable to an associated tube for providing a selected flow of diluted NO-containing air to a patient.

A method is disclosed for providing a NO-containing gas flow to treat a biological object. The method may include: receiving NO-rich air from a plasma-generated NO source; directing the NO-rich air to a scrubber to remove NO₂ from the NO-rich air; diluting the scrubbed NO-rich air with atmospheric air to obtain air having a targeted concentration of NO; and the diluted NO-containing air to a plurality of patient locations.

The method may further include cooling a solvent disposed in the scrubber, where cooling the solvent comprises pumping the solvent through a heat exchanger. The method may also include dehumidifying the scrubbed NO-rich air discharged from the scrubber. The method may include measuring a concentration of NO in the diluted NO-containing air in the manifold. The method can include adjusting the gas mixture based on the measured NO concentration e to adjust an amount of atmospheric air to mix with the scrubbed NO-rich air.

The method may include providing a selected flow of diluted NO-containing air to a patient. In some embodiments, the NO-rich air from the plasma-generated NO source may be provided at a flow rate of from 1 to 10 L/min and a temperature of from 300 to 1000° C. In some embodiments, the scrubbed NO-rich air may be provided at a flow rate of from 1 to 10 L/min and a temperature of from 25 to 40° C. In some embodiments, the dilute NO-containing air is provided at a flow rate of up to 1200 L/min and at ambient room temperature.

The disclosed system relies on the NO gas provided by a plasmatron or similarly powerful NO generator, which allows the system to output therapeutic volumes of nitric oxide not to just one but to multiple patients, up to a hundred (depending on required therapeutic concentration of NO). As such, deployment of one such system in a hospital may alleviate the need for multiple, often compressed-gas-cylinder-based nitric oxide delivery systems, which can result in significant cost and space saving. Also, the disclosed system requires only electricity as a consumable energy supply, and thus it can be rapidly deployed in the areas of the world where access to specialty compressed gases, like NO, is limited.

### BRIEF DESCRIPTION OF THE DRAWINGS

By way of example, specific embodiments of the disclosed device will now be described, with reference to the accompanying drawings, in which:

FIG. 1 is a schematic view of a first embodiment of a device for conditioning plasma-generated NO to provide a flow of NO-containing gas for treatment of patients;

FIGS. 2A-2D are isometric, and first, second and third cross-section views of a high-performance mixer for use in conditioning plasma-generated NO to provide a flow of NO-containing gas for treatment of patients; and

FIG. 3 is a schematic view of a second embodiment of a device for conditioning plasma-generated NO to provide a flow of NO-containing gas for treatment of patients.

### DETAILED DESCRIPTION

Potential benefits from treating patients with lung infections with plasma-generated NO include control of the bacteria and/or virus, reduction in respiratory tract inflammation, reduction in respiratory tract infection, and an increase in respiratory tract oxygenation.

Inhalation

There is widespread interest in the use of NO for pulmonary ailments. NO is currently approved for the treatment of pulmonary hypertension in neonates in the INOmax therapy, manufactured by Mallinckrodt Pharmaceuticals: https://www.inomax.com/. Serious questions remain, however, as to whether NO can penetrate the mucus and congestion found in the airways in certain disease states at least in sufficient quantities to have a therapeutic effect. These doubts are not present in relation to the upper respiratory system. However, the limitations described below have restricted the concentration of NO to levels where its effect is either reduced or non-existent. Under the inventors' proposed approach, higher concentrations should be permissible.

Method of Administration

The disclosed system can “condition” a high-temperature high-concentration plasma-generated stream of NO-rich air to cool down the air and remove impurities, primarily excessive nitrogen dioxide (NO₂). Following conditioning, the disclosed system can provide a variable (10-2000 parts per million (ppm)) concentration and flow rate (0.1-10 standard liters per minute (SLPM)) of NO via industry-standard connections compatible with inhalation and intubation systems currently in use.

Limitations on the Use of Inhaled NO

The Food and Drug Administration (FDA) currently regulates the concentration (ppm) of NO that can be inhaled to ensure safety of the patient. The concentration of NO shown to be useful in treating hypertension in infants is below 50 ppm. The principal risk in providing high dosages of NO to patients is the presence of NO₂ within the stream. In addition, current NO supply systems are limited to delivering NO to a single patient at a time. The inventors' approach is designed to address this problem and to allow higher concentrations of NO to be delivered to patients than under conventional methods of delivery. The inventors' approach is also designed to allow NO to be delivered to multiple patients simultaneously.

### INTRODUCTION

In all the cases of use of nitric oxide (NO) for inhalation or other medical uses resulting in direct contact of NO with lung tissue (including all the organs on the way to lungs), it is of great importance to remove nitrogen dioxide (NO₂), which is ever-present due to the continuous reaction of NO with oxygen (O₂), which is indispensable for breathing. NO₂ generation (see Equation (1)) is strongly dependent on NO concentration and does not depend on temperature, as it has a negative activation energy of −4.41 kJ/mol. Therefore, it is crucial in some systems, such as IonoJet, which are capable of producing very high concentration (over 2% by volume or 20,000 ppm) of NO, to reduce that concentration to consumption level, which currently FDA has approved at 20 ppm.

O₂+NO+NO→NO₂+NO₂ (1)

The secondary issue pertinent to systems producing NO at very high concentrations is the temperature of the air containing that NO, because usually such high concentrations of NO are achieved using arc discharge plasmas. Depending on design, such systems may heat the entire volume of air passing through the NO generator to a temperature of over 1000 degrees Celsius (° C.). Air at such temperature is unusable in clinical air mixing systems, which are commonly designed to operate with room temperature air.

Device Design

The inventors have developed a NO distribution system for arc-based NO generators, such as IonoJet and PLASON, which solves both of the above-identified problems. The system 1, shown in Error! Reference source not found., accepts hot NO-rich air (A1) (concentration ranging from 1,000 ppm to 50,000 ppm) from a generator 2 via “nozzle receptacle” port 4 using custom “adapter bushing” 6 that enables connection to nozzles from different systems. In some embodiments the flow of hot NO-rich air may be provided at a flowrate of 1-10 liters/minute (L/min) and a temperature of from 300 to 1000° C. Following the injection of the entire NO/air mixture, inescapably containing NO₂ (up to 0.5% by volume or 5000 ppm), the mixture is directed via tubing 8 (metal, in one embodiment stainless steel) into an NO₂ scrubber/gas washing bottle 10, made from either high temperature glass or metal. The scrubber 10 can contain NO₂ absorbing liquid (referred to as a “solvent” (S), including but not limited to distilled water and low concentration alkaline solutions). In the particular case where distilled water is used as a scrubbing liquid, NO₂ hydrolysis in water forms nitrous (HNO₂) and nitric acid (HNO₃), which are completely soluble with former converting to water at higher temperatures and/or concentrations with release of NO (see Equation 2).

3HNO₂→HNO₃+2NO+H₂O (2)

The hot air (A2) passing through the scrubber 10 cools down to a temperature slightly above room temperature (in one non-limiting example by 10° C.). Such cooling it heats up the solvent (S) reducing its effectiveness. To prevent heat accumulation the solvent (S) can be continuously recirculated through a heat exchanger 12 using pump 14, and then returned to the scrubber 10. In one non-limiting example the heat exchanger 12 is a fan assisted radiator. The cooling system 1 can also include pH, conductivity, and temperature sensor(s) (not shown) to enable motoring of solvent quality. The cooling system can also include ports (not shown) for draining and refilling. In some embodiments the scrubber 10 can have a volume of from 0.5 to 5 liters, and in one particular embodiment the scrubber 10 has a volume of about 1 liter. In some embodiments, the flow of scrubbed NO-rich air “A2” may be provided at a flowrate of 1-10 L/min and a temperature of from 25 to 40° C.

After the scrubber 10, NO-rich air “A2” may need to be dehumidified, and therefore, an optional condenser 16 can be used. Such condenser 16 can operate either as batch type device refilled with ice, dry ice, or liquid nitrogen, or it may be continuously liquid cooled with either specialized refrigerant or water.

Following moisture removal, the NO-rich air (A2) can be diluted to a target concentration, which may be determined from the inhaled/intubated NO concentration (specified by user but within range of 20 ppm to 2000 ppm). The dilution is performed using a flow of synthetic or atmospheric air (A3) from an external compressor. A gas mixer 18, which in the illustrated embodiment is a Venturi injector, can be used to inject low flow rate NO-rich air into high flow rate dilution air. In some embodiments, the air (A3) from the external compressor may be provided to the gas mixer 18 at a flow rate of from 120-1200 L/min, and in one embodiment may be provided at a flow rate of 500 L/min. Target NO concentration is monitored at manifold 19 using a concentration monitor 20, and the amount of external air supply A3 can be varied using the concentration monitor 20 for continuous feedback. Based on the feedback from the concentration monitor 20, the gas mixer 18 may adjust the amount of atmospheric air A3 to add to the NO-rich air (A2). In some embodiments, the resulting mixed NO-containing air A4 can be provided at up to 1200 L/min and at ambient temperature.

The resulting air A4 is large in volume but low in NO concentration and should be used immediately. For this purpose, the system 1 includes distribution manifold 19 to provide the ability to connect multiple heart-lung machines or inhalation or intubation devices (patient #1-patient #n) via individual valves or flow controllers 22. The manifold outlet ports should all be independently switchable ON or OFF, and the flow rate from each should be adjustable by the user, independently from each other, to suit a specific treatment. It is expected that not all the NO-containing air will be used for treatments, and therefore the excess air A5 is exhausted out of the system.

In example embodiments, the manifold is always open to dump excess air A5 and does not hold the NO-containing air therein. Thus, in some embodiments the manifold 19 may be coupled to individual valves with blowers rather than to individual flow controllers 22. The exhaust A5 can be connected directly to a suitable ventilation system and not exhausted into the room.

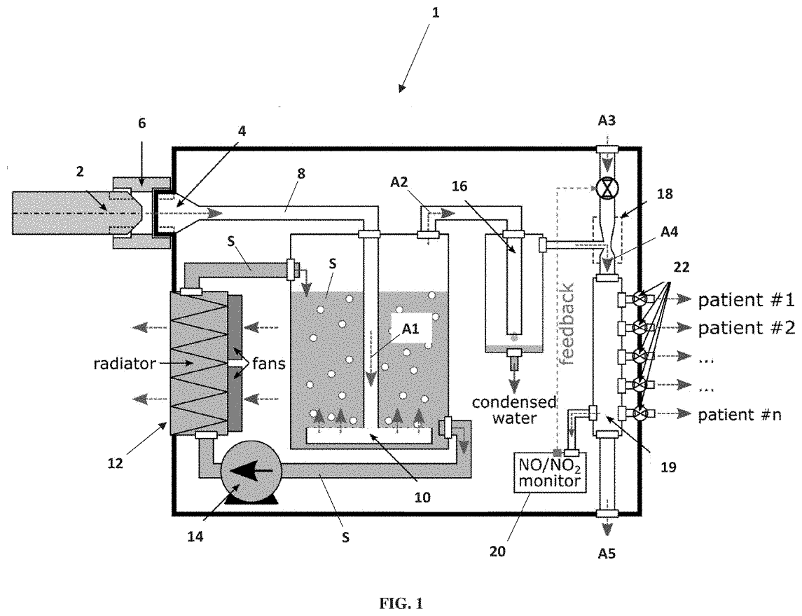

As previously discussed, a primary objective of the present inventions is both to cool down and dilute NO to prevent fast NO-to-NO₂ conversion at high concentrations of NO. An arrangement for achieving this objective can be to mix the exhaust of the nozzle of an arc-based NO generator 2 (see FIG. 1) with a large volume of relatively cold (e.g., ambient temperature) air. Such arc-based NO generators collectively are called plasmatrons-(gliding-)arc plasma generators, where plasma is stabilized by the flow of gas.

While exhausting overheated NO-rich air directly into a room environment may drop the temperature and dilute the NO concentration, it also that creates a strongly non-uniform distribution of NO concentration, which requires that the treatment region be positioned at a specific distance from the nozzle.

Moreover, in such a configuration there can be no effective control of NO concentration. For example, it is not possible to “dial-in” a specific NO concentration (e.g., 20, 100, or 1000 ppm), each of which may be used for different treatments regimens, from inhalation to skin and wound applications. Such configurations also allow no control over the generation of NO₂, which is distributed unevenly not only by magnitude, but also by the ratio of its concentration to the concentration of NO.

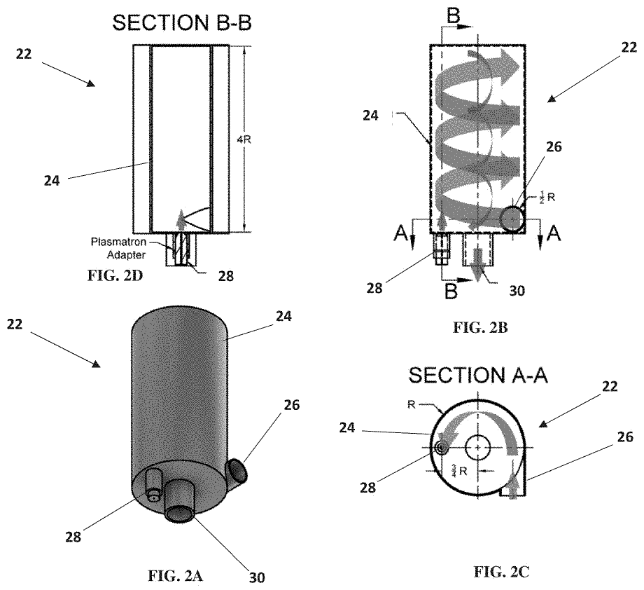

To address these issues, and referring now to FIGS. 2A-2D, the inventors have developed an advanced gas-mixing device 22 which dilutes NO and cools it to a temperature suitable for inhalation by a patient. The resulting NO-containing air is also uniformly mixed to a desired concentration. The disclosed device 22 is designed to receive NO-containing gas from arc-discharge plasmatrons (e.g., generator 2 in FIG. 1) with flow parameters of from about 1 to 10 L/min of exhaust, and with average temperatures from 200 to 1000° C.

Features of the gas-mixing device include a large flow rate ratio between relatively cold (e.g., ambient temperature) air, and the exhaust from the generator 2, which are combined in the gas-mixing device 22 by employing a reverse vortex mixing chamber.

The flow ratios (i.e., volume flow ratios of ambient air to generator exhaust) required for efficient mixing are from 20:1 to 2000:1. Ratios lower than 20:1 are not believed to provide sufficient dilution or drop in temperature. Ratios over 2000:1 approach the limit of NO usability as at expected that an initial concentration of NO of 2%-3% (20000 ppm-30000 ppm) dilution of 2000 times would create a flow with less than 20 ppm of NO, which is less than necessary for existing effective inhalation treatments.

The disclosed gas mixing device 22 is illustrated in FIGS. 2A-2D. The device 22 includes a chamber 24 having two gas inlets: a tangential large diameter inlet 26 for diluting air and a plasmatron inlet 28, whose axis is oriented parallel to the central axis of the chamber 24 and offset from it by a distance “Dl”, which in one non-limiting example is about ¾ of the chamber radius (R). The chamber 24 can have a height (or length, largest physical dimension) that in one embodiment is equal to 4×R. A single gas outlet 30 is positioned coaxially with the central axis and is disposed on the side of the chamber 24 that is adjacent to the tangential inlet 26 and is on the same side of the chamber 24 as the plasmatron inlet 28. In the illustrated embodiment, this side can be referred as bottom of the chamber 24, and the opposing side of the chamber (i.e., the top) has no inlets or outlets. The diameter of the gas outlet 30 is selected such that its cross-sectional area is 1.5 to 2 times the size of the area of the tangential inlet 26. The unique physical properties of reverse vortex flow and their benefits are well known in the art, and thus will not be described in greater detail herein.

It will be appreciated that it is also possible to use a forward vortex configuration for the mixing chamber 24, where the outlet is positioned at the top in lieu of the bottom. Such a configuration, while not as efficient as the illustrated reverse vortex configuration, may provide sufficient mixing and dilution, especially at high flow ratios, and also provides the flexibility of having outlet on the opposite side of the chamber, which may reduce potential interference issues with inlets on the bottom.

Although not shown, the gas outlet 30 can be coupled to a distribution manifold (similar to manifold 19 of FIG. 1) to provide the ability to connect multiple heart-lung machines or inhalation or intubation devices (similar to patient #1-patient #n via individual valves or flow controllers 22 as shown in FIG. 1). The manifold outlet ports should all be independently switchable ON or OFF, and the flow rate from each should be adjustable by the user, independently from each other, to suit a specific treatment.

In addition, target NO concentration may be monitored at the manifold using a concentration monitor (similar to concentration monitor 20 of FIG. 1), and the amount of external air supply provided via tangential inlet 26 can be varied using the concentration monitor 20 for continuous feedback. Based on the feedback from the concentration monitor 20. In some embodiments, the resulting mixed NO-containing air dispensed through outlet 30 can be provided at up to 1200 L/min and at ambient temperature.

The reverse vortex mixing chamber 24 provides extremely efficient convectional cooling and mixing. This, combined with the effect of a nozzle (plasmatron inlet 28) exhausting immediately into the chamber 24 provides a maximum quenching rate for the reaction (1) as the dilution 1000 (10³) fold will drop the reaction rate by 10⁶ times. In this way, the reverse vortex chamber primarily prevents NO₂ formation at the core (keeping in mind that at the core the concentration of NO₂ is close to 0 as all the gases in arc plasma are in dissociated state).

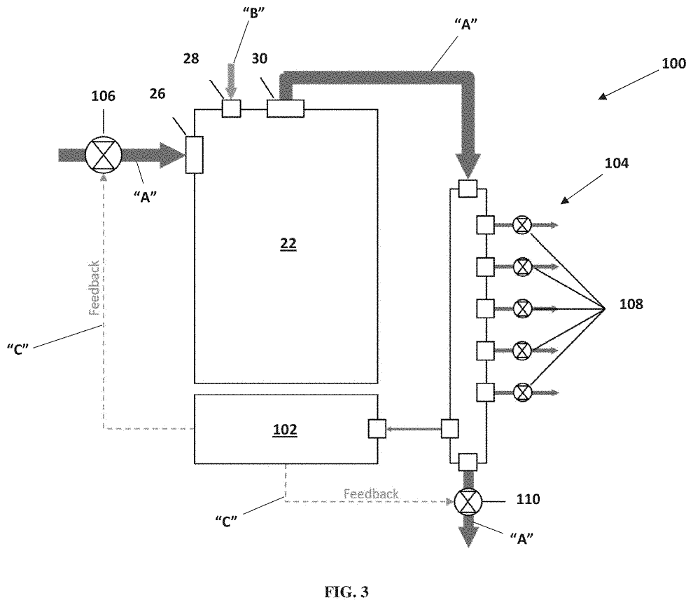

FIG. 3 illustrates another embodiment of a NO distribution system 100 for arc-based NO generators which incorporates the reverse vortex mixer 22 of FIGS. 2A-2D in lieu of the water scrubber 10 of FIG. 1. The system 100 of FIG. 3 includes a diluting air inlet 26, a plasmatron inlet 28, a mixed gas outlet 30, a monitoring and control subsystem 102 for monitoring and control of NO, NO₂, pressure and temperature. A therapeutic gas distribution manifold 104 is provided that is similar or the same as the manifold 19 described in relation to FIG. 1. An optional diluting air flow rate controller 106 may be used to receiving feedback from the control subsystem 102. A plurality of therapeutic gas flow controllers 108 may be used to disturbed therapeutic gas to up to “n” patients. A waste air valve/controller 110 may be used to maintain stable pressure in the therapeutic gas distribution manifold 104.

Arrows “A” indicate direction and relative magnitude of the gas flows (not to scale), while arrow “B” highlights high-temperature and high-concentration intake from a plasmatron. Arrows “C” illustrate electrical communications.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

While certain embodiments of the disclosure have been described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A system for providing a NO-containing gas flow to treat a biological object, the system comprising:
a nozzle receptacle for receiving NO-rich air from a plasma-generated NO source;
tubing coupled to the nozzle for directing the NO-rich air to a scrubber, the scrubber configured to receive a solvent for absorbing NO₂;
tubing coupled between the scrubber and a gas mixer for directing scrubbed NO-rich air to the gas mixer,
the gas mixer further coupled to a source of atmospheric air for selectively mixing the scrubbed NO-rich air with the atmospheric air to create diluted NO-containing air; and
a manifold for distributing the diluted NO-containing air to a plurality of patient locations.

2. The system of claim 1, further comprising a cooling loop for cooling the solvent in the scrubber, and wherein the cooling loop comprises a pump and a heat exchanger.

3. (canceled)

4. The system of claim 1, further comprising a condenser for dehumidifying the scrubbed NO-rich air discharged from the scrubber.

5. The system of claim 1, further comprising a concentration monitor coupled to the manifold for measuring a concentration of NO in the diluted NO-containing air in the manifold; and a feedback loop coupled between the concentration monitor and the gas mixer, the feedback loop for adjusting the gas mixture to adjust an amount of atmospheric air to mix with the scrubbed NO-rich air.

6. (canceled)

7. The system of claim 1, further comprising a plurality of valves coupled to the manifold, each of the plurality of valves couplable to an associated tube for providing a selected flow of diluted NO-containing air to a patient.

8-20. (canceled)

21. A system for providing a NO-containing gas flow to treat a biological object, the system comprising:
a gas mixing device comprising:
a mixing chamber for reverse vortex mixing of atmospheric air and NO-rich air,
a tangential air inlet for directing atmospheric air into the mixing chamber,
a nozzle receptacle for directing NO-rich air from a plasma-generated NO source into the mixing chamber; and
an outlet for dispensing diluted NO-containing air from the gas mixing chamber; and
a manifold coupled to the outlet of the gas mixing device for receiving the diluted NO-containing air and for distributing the diluted NO-containing air to a plurality of patient locations.

22. The system of claim 21, wherein flow ratios of the atmospheric air to the NO-rich air are from 20:1 to 2000:1.

23. The system of claim 21, wherein the nozzle receptacle has an axis oriented parallel to a central axis of the mixing chamber, and wherein the nozzle receptacle is offset from the central axis of the mixing chamber by a distance equal to four times a radius of the mixing chamber.

24. (canceled)

25. The system of claim 21, wherein the outlet is coaxial with the central axis of the mixing chamber, and wherein the outlet is positioned on a side of the mixing chamber adjacent to the tangential air inlet, and the outlet is position on the same size of the mixing chamber as the nozzle receptacle.

26. (canceled)

27. The system of claim 25, wherein a diameter of the outlet is 1.5 to 2 times a size of the tangential air inlet.

28. The system of claim 21, further comprising a concentration monitor coupled to the manifold for measuring a concentration of NO in the diluted NO-containing air in the manifold, and a feedback loop coupled between the concentration monitor and the gas mixing device, the feedback loop for adjusting the gas mixture to adjust an amount of atmospheric air to mix with the NO-rich air.

29. The system of claim 21, where the NO-rich air from the plasma-generated NO source is provided at a flow rate of from 1 to 10 L/min and a temperature of from 300 to 1000° C.

30. The system of claim 21, wherein the dilute NO-containing air is provided at a flow rate of up to 1200 L/min and at ambient temperature.

31. A method for providing a NO-containing gas flow to treat a biological object, the method comprising:
receiving, in a mixing chamber, atmospheric air through a tangential air inlet,
receiving, in the mixing chamber, NO-rich air from a plasma-generated NO source through a nozzle receptacle; and
reverse vortex mixing of the atmospheric air and the NO-rich air in the mixing chamber,
dispensing diluted NO-containing air from the gas mixing chamber; and
distributing the diluted NO-containing air to a plurality of patient locations.

32. The method of claim 31, wherein flow ratios of the atmospheric air to the NO-rich air are from 20:1 to 2000:1.

33. The method of claim 31, wherein the nozzle receptacle has an axis oriented parallel to a central axis of the mixing chamber, and wherein the receptacle is offset from the central axis of the mixing chamber by a distance equal to four times a radius of the mixing chamber.

34. (canceled)

35. The method of claim 31, wherein the outlet is coaxial with the central axis of the mixing chamber.

36. The method of claim 35, wherein the outlet is positioned on a side of the mixing chamber adjacent to the tangential air inlet, and the outlet is positioned on the same size of the mixing chamber as the nozzle receptacle.

37. The method of claim 35, wherein a diameter of the outlet is 1.5 to 2 times a size of the tangential air inlet.

38. The method of claim 31, further comprising monitoring a concentration of NO in the diluted NO-containing air and adjusting the gas mixture to adjust an amount of atmospheric air to mix with the NO-rich air.

39-42. (canceled)
